(19) United States
(12) Patent Application Publication
(10) Pub. No.: US 2005/0043412 A1
(43) Pub. Date: Feb. 24, 2005

(54) Remedies for glaucoma comprising bunazosin and prostaglandins

(75) Inventors: Masaki Ichikawa, Ikoma-shi (JP); Fumio Nakazawa, Ikoma-shi (JP); Hideaki Hara, Ikoma-shi (JP)

Correspondence Address: FRISHAUF, HOLTZ, GOODMAN & CHICK, PC, 767 THIRD AVENUE, 25TH FLOOR, NEW YORK, NY, 10017-2023, US

(73) Assignee: SANTEN PHARMACEUTICAL CO LTD

(21) Appl. No.: 10/503,031

(22) PCT Filed: Jan. 28, 2003
(86) PCT No.: PCT/JP03/00786

(30) Foreign Application Priority Data
Jan. 29, 2002 (JP) 2002-020542

(51) Int. Cl. A61K 31/557; A61K 31/551

(52) U.S. Cl. 514/573; 514/218

(57) ABSTRACT

The object of the present invention is to find utility of a combination of bunazosin, having a new action mechanism, and prostaglandins as a therapeutic agent of glaucoma. The invention relates to a therapeutic agent of glaucoma, comprising bunazosin or a salt thereof and prostaglandins in combination, where bunazosin or a salt thereof and prostaglandins mutually supplement and/or enhance each action, and where bunazosin and prostaglandin may be formulated in a single preparation to be administered or may be separately administered in combination.

### TECHNICAL FIELD

The present invention relates to a therapeutic agent of glaucoma, the therapeutic agent comprising bunazosin or a salt thereof and prostaglandins in combination.

### BACKGROUND OF THE INVENTION

Glaucoma is an intractable ocular disease with a risk of blindness, involving the increase of intraocular pressure due to various factors. Studies have been done about various treating methods therefor. The method for lowering intraocular pressure includes three approaches, namely pharmacotherapy, laser therapy and surgery. For the pharmacotherapy, drugs such as β-blockers, prostaglandin-related drugs, carbonic anhydrase inhibitors, cholinergic agents, and epinephrine-related drugs have been used.

Lately, bunazosin hydrochloride, an α1 blocker, was developed as a drug based on a new action mechanism. Bunazosin hydrochloride is a selective blocker of sympathetic nerve α1 receptor and promotes the uveoscleral outflow to thereby lower intraocular pressure (Folia Ophthalmologica Japonica, 46, 1066-1070, 1995).

As a combination of al blocker and another therapeutic agent of glaucoma, Japanese Patent Laid-Open Publication (hereinafter referred to as JP-A) 2001-33551 describes a therapeutic agent of glaucoma comprising an al blocker and an angiotensin II antagonist.

Meanwhile, Japanese Patent No. 2726672 states a combination of an adrenergic receptor blocker and prostaglandins, as a therapeutic agent of glaucoma comprising a combination of prostaglandins and another therapeutic agent. However, this Japanese patent has no description about the effect of a blockers because β blockers in this patent are mainly adrenergic blockers. In particular, this Japanese patent does not even suggest the effect of al blocker.

As described above, no report has been issued about the effect on treating glaucoma by a combination of an al blocker and prostaglandins. In particular, studies about bunazosin, an al blocker having a novel action mechanism, in combination with prostaglandins have never been done.

Accordingly, it has been a very interesting subject matter to find utility of a combination of bunazosin having a new action mechanism and prostaglandins as a therapeutic agent of glaucoma.

### DISCLOSURE OF THE INVENTION

The present inventors made intensive studies about the possibility of the development of a therapeutic agent of glaucoma by combining bunazosin with prostaglandins. Consequently, the inventors found that the combination thereof enhanced the action of lowering intraocular pressure, thereby completing the present invention. The detailed test method and the results thereof are described below in the section of Pharmacological Test. Bunazosin in combination with prostaglandins exhibited a remarkable action of lowering intraocular pressure. Additionally, the therapeutic agent of glaucoma in accordance with the present invention can be used preferably not only for the treatment of glaucoma but also for the prevention thereof.

The present invention relates to a therapeutic agent of glaucoma comprising bunazosin or a salt thereof and prostaglandins in combination. These drugs mutually supplement and/or enhance their actions.

For the treatment of glaucoma, bunazosin and prostaglandins may be formulated in a single preparation to be administered. In other words, these drugs may be administered in mixture. Alternatively, each drug may be in a separate preparation and these drugs may be administered in combination.

Bunazosin can be in the form of its salt. Examples of the salts include salts thereof with inorganic acids such as hydrochloric acid and nitric acid. In particular, the hydrochloride salt is preferable.

Since the present invention is characterized by treating glaucoma with bunazosin and prostaglandins in combination, any prostaglandins having the action of lowering intraocular pressure and utility in treating glaucoma may be used, with no specific limitation. Prostaglandins having the action of lowering intraocular pressure are exemplified by prostaglandins described in JP-A-Sho59-1418 (natural prostaglandins, particularly prostaglandin F2α), prostaglandins such as latanoprost as described in Published Japanese Translation of PCT No. 3-501025, prostaglandins such as isopropyl unoprostone as described in JP-A-Hei2-108, prostaglandins such as bimatoprost as described in Published Japanese Translation of PCT No. 8-501310, and prostaglandins such as travoprost as described in JP-A-Hei10-182465. In particular, latanoprost, isopropyl unoprostone, bimatoprost or travoprost, which has already been on the market as a therapeutic agent of glaucoma, is preferably used. It is needless to say that these prostaglandins may be in salt forms or ester forms thereof.

According to the embodiment of the invention, the formulation can be either one formulation containing bunazosin and prostaglandins in mixture or two separate formulations containing each component. Not any specific technique is needed for the preparation of these formulations. They are prepared by widely used methods. Preferably, these formulations are topically administered to eyes. The dosage forms are exemplified by eye drops and eye ointments.

The separate formulations containing bunazosin and prostaglandins respectively can be prepared according to known methods. They are exemplified by the formulation disclosed in Japanese Patent Publication No. 2610619, the formulation disclosed in Japanese Examined Patent Publication Hei 7-23302, and commercially available formulations. The formulation of prostaglandins can be prepared with reference to the descriptions of the above-mentioned Japanese patent laid-open publications. Commercially available formulations of latanoprost, isopropyl unoprostone and the like as glaucoma-treating agents can be used.

The formulation containing bunazosin and prostaglandins in mixture can be also prepared according to known methods. The eye drops can be prepared, using isotonic agents such as sodium chloride and concentrated glycerin; buffers such as sodium phosphate buffer and sodium acetate buffer; surfactants such as polyoxyethylene sorbitan monooleate, stearate polyoxyl 40, and polyoxyethylene hardened castor oil; stabilizers such as sodium citrate and sodium edetate; and preservatives such as benzalkonium chloride and paraben, as needed. The pH should be within an ophthalmologically acceptable range and is preferably within a range of pH 4 to pH 8. For reference, a formulation example thereof is described below in the section of Example. However, the formulation example never limits the scope of the invention.

The doses of bunazosin and prostaglandins can be determined depending on the symptom and age of patients, the dosage form, the administration route and the like. In case of an administration through eye drops, for example, bunazosin is administered generally within 2 to 40 μg daily from once to several times a day. The dose of prostaglandins varies depending on the prostaglandin type. The dose can be determined on the basis of the actual dose range for treatment and is raised or lowered depending on the symptom of patients and the like. The daily dose is within a range of 1 to 1,000 μg, which is administered from once to several times a day. More specifically, isopropyl unoprostone and latanoprost are generally administered at a daily dose of 30 to 300 μg and a daily dose of 1 to 5 μg, respectively. Depending on the symptom of patients and the like, the doses are varied. Based on similar standards, the doses of other prostaglandins can be determined. These doses are also applicable to the administration of bunazosin and prostaglandins in combination. In case that bunazosin and prostaglandins are to be administrated in one formulation, the formulation should be prepared by selecting the mixing ratio of two drugs appropriately so that their daily doses might not excess each dose of the separate drugs. The mixed formulation is administered from once to several times daily.

A formulation example and a pharmacological test are shown in the following Example. The Example is for better understanding of the invention but never limits the scope of the invention.

### BEST MODE FOR CARRYING OUT THE INVENTION

[Formulation Example]

A general formulation example of eye drops containing bunazosin and prostaglandins in mixture in accordance with the invention is described below.

Eye drops (in 100 mL)

| | |
|---|---|
| Bunazosin hydrochloride | 0.01 g |
| Latanoprost | 0.005 g |
| Boric acid | 0.5 g |
| Concentrated glycerin | 2.0 g |
| Benzalkonium chloride | 0.01 g |
| Dilute hydrochloric acid | q.s. |
| Distilled water | q.s. |

[Pharmacological Test]

So as to study the utility of bunazosin in combination with prostaglandin, bunazosin and prostaglandin were administered in combination to cynomolgus monkeys (Macaca fascicularis), examining the effect on intraocular pressure. Latanoprost was used as prostaglandin.

(Test Compound Solution)

For bunazosin administration, Detantol (trade mark) ophthalmic solution, which contains bunazosin hydrochloride at 0.01%, was used. For latanoprost administration, Xalatan (trade mark) ophthalmic solution, which contains latanoprost at 0.005%, was used.

(Dosing Groups)

Cynomolgus monkeys were divided into four groups, a group to be dosed with a vehicle (vehicle group), a group to be dosed with bunazosin (bunazosin group), a group to be dosed with latanoprost (latanoprost group) and a group to be dosed with bunazosin and latanoprost [(bunazosin+latanoprost) group].

(Administration Method and Measurement Method)

1. For topical anesthesia, a drop of 0.4% oxybuprocain hydrochloride ophthalmic solution was instilled into a single eye of each normal cynomolgus monkey. Then, intraocular pressure was measured before drug administration.
2. Then, each test compound solution was administered. To the vehicle group, 20 μl of the vehicle solution was instilled. To the bunazosin group, 20 μl of Detantol ophthalmic solution was instilled. To the latanoprost group, 20 μl of Xalatan ophthalmic solution was instilled . To the (bunazosin+latanoprost) group, 20 μl of Detantol ophthalmic solution was instilled and 5 minutes later 20 μl of Xalatan ophthalmic solugion was instilled.
3. 2, 4 and 6 hours after the administration of these test compound solutions, one drop of 0.4% oxybuprocain hydrochloride eye drops was instilled for topical anesthesia, and the intraocular pressure was measured. The maximum reduction of intraocular pressure in each group was determined by the following formula. (Maximum reduction of intraocular pressure)=(intraocular pressure at the time when the intraocular pressure was decreased mostly)−(intraocular pressure before drug administration)
   (Results and Discussion)

The experimental results are shown in Table 1.

TABLE 1

| | Maximum reduction of |
|---|---|
| | intraocular pressure (mmHg) |
| Vehicle group | 0.9 |
| Bunazosin group | 1.9 |
| Latanoprost group | 2.6 |
| Bunazosin + latanoprost group | 3.0 |

As shown in Table 1, the intraocular pressure in the group dosed with bunazosin and latanoprost was lowered more than those in the bunazosin group and the latanoprost group. Those described above show that the combination of bunazosin and prostaglandins can bring about a remarkable reduction of intraocular pressure.

Industrial Applicability

The administration of bunazosin and prostaglandins in combination enhanced the intraocular pressure-lowering effect of each drug. Thus, the pharmaceutical composition of the invention is useful as a therapeutic agent of glaucoma.

1. A therapeutic agent of glaucoma, comprising bunazosin or a salt thereof and a prostaglandin in combination.

2. A therapeutic agent of glaucoma, comprising bunazosin or a salt thereof and a prostaglandin in combination, where these drugs mutually supplement and/or enhance the actions thereof.

3. A therapeutic agent of glaucoma according to claim 1 or 2, where the prostaglandin is latanoprost.

4. A method for treating glaucoma, comprising administration to a patient with a therapeutically effective amount of bunazosin or a salt thereof and a prostaglandin in combination.

5. A method for treating glaucoma according to claim 4, where the prostaglandin is latanoprost.
